(19) United States
(12) Patent Application Publication
(10) Pub. No.: US 2014/0088445 A1
(43) Pub. Date: Mar. 27, 2014

(54) Adhesive Transducer Holder

(71) Applicant: Daniel James Albershardt, Mercer Island, WA (US)

(72) Inventor: Daniel James Albershardt, Mercer Island, WA (US)

(73) Assignee: Daniel Albershardt, Mercer Island, WA (US)

(21) Appl. No.: 13/787,248

(22) Filed: Mar. 6, 2013

Related U.S. Application Data
(60) Provisional application No. 61/609,355, filed on Mar. 11, 2012.

(51) Int. Cl. A61B 5/021

(52) U.S. Cl. CPC A61B 5/021
USPC 600/485

(57) ABSTRACT

System, methods and apparatus for a disposable transducer mounting device that secures one transducer to a patient. Based on the illustrative embodiments, the holder is of rigid or semi-rigid material that has the ability to secure the transducer via rails and has an adhesive base that is used to secure the transducer and mounting device to the patient at the desired location.

### BACKGROUND

In the hospital operating facilities and recuperating settings, it is often necessary for medical health professionals to monitor the blood pressure, cardiac output, and central venous pressure (CVP) of patients. When minute-to-minute and even beat-to-beat monitoring is required, many medical health professionals measure those parameters with a pressure transducing system. This would involve placing an invasive single or multiple lumen catheter into either the arterial or venous system of the patient. If the catheter is placed in the peripheral arterial system, it can measure the blood pressure and mean arterial pressure. When the catheter is placed in a large central vein, it can be used to measure the CVP. When the catheter is placed in the pulmonary artery, it can used to measure the pulmonary artery pressure or the pulmonary artery wedge pressure. Measurement of all of these various data sources involve connecting a host of different equipment to the patient comprising of: plastic tubing, a transducer, a bag of appropriate intravenous fluid that is pressurized, and a computer connection cable. The plastic tubing connects the indwelling catheter, the transducer and the intravenous fluid reservoir via plastic connectors. The transducer is typically housed in plastic and converts fluid pressure into an electronic signal that is displayed on a monitor for the medical health professional to interpret. The measurement data from the transducer is not clinically useful until the system has been calibrated via a process that is often termed ‘zeroing’ to obtain absolute measurement data. Zeroing involves physically aligning the tip of the indwelling catheter or the desired reference point (i.e., the heart, which is approximated by the 4th intercostal space at the mid-axillary line) with the transducer stopcock assembly.

There are two basic methods for zeroing, also known as ‘leveling’, the transducer. The first option is to affix the transducer to a pole next to the patient bed or operating table at the appropriate height such that it is level with the desired reference point. A laser level is used to ensure level placement. However, whenever the bed height is changed or the patient's position is changed, the ‘zero’ is altered and the system must be re-leveled. If it is not re-leveled and zeroed, the system will report inaccurate pressure readings.

The other basic method is to place the transducer at the desired location on the arm or chest of a patient. The transducer is then affixed using a Velcro® strap or medical tape, thus eliminating the need to re-zero the system after the operating table is moved or the patient elevation is changed. Straps can be difficult to apply on anesthetized or non-compliant patients leading to the straps being insecure or loose, causing mal-placement of the transducer. Medical tape is difficult to apply to transducers in such a way that it will securely retain the transducer in the proper position and withstand the force necessary to open the flush valve of the transducer. This may cause mal-positioning of the transducer with manual handling and can lead to the transducer becoming loosened further, contributing to inaccurate pressure readings.

### SUMMARY OF THE INVENTION

System, methods and apparatus for the disposable transducer mounting device, or holder, that is designed to secure one pressure transducer to the patient or other surface. It is of rigid or semi-rigid material that has a base and rails for the transducer to slide into place and be held securely. The base of the holder has adhesive material that is designed to be affixed to the desired surface (e.g. the patient, bed, table, or other surface). The transducer may be removed and replaced in the holder many times without degrading the absolute positioning of the transducer.

### BRIEF DESCRIPTION OF THE DRAWINGS

FIG. 1: Plan view of the transducer holder
11 Base of the transducer holder
12 Rails that guide and hold the transducer in place
13 Tapered tip of the rail to facilitate ease of placement of transducer into the holder
14 Rails that support the bottom of the transducer and prevent suction-lock of the transducer to the holder if it comes in contact with water. Upper end of the rails are tapered to provide ease of placement of the transducer into the holder

FIG. 2: Elevation view of the transducer holder
21 Base of the transducer holder
22 Rails as described in FIG. 1: 12, showing the endplate of the rails that locks the transducer in place
23 Rails as described in FIG. 1: 14
24 Orientation of adhesive that is applied to the base

FIG. 3: Illustrative depiction of the transducer being fitted into the holder

FIG. 4: Illustrative depiction of a sample application point of the holder
41 The transducer holder
42 The heart
43 The invasive catheter
44 The output monitor

### DETAILED DESCRIPTION OF THE DRAWINGS

The following detailed description of the invention will make reference to the drawings represented in FIGS. 1 through 4. The disposable transducer mounting device will be made of rigid or semi-rigid material that consists of multiple sub-components.

Referring to FIG. 1, the base, 11 & 21, is shown in these drawings to have a slightly curved shape, 21, to allow for ergonomic placement on a curved surface, e.g. an arm. However the base can be made flat. The disposable transducer would be guided and held in place by the side rails, 12. To prevent vacuum lock of the transducer in the holder, two separate rails, 14, elevate the transducer above the base, 11, of the holder.

Referring to FIG. 2, the adhesive backing of the transducer holder would be at 24.

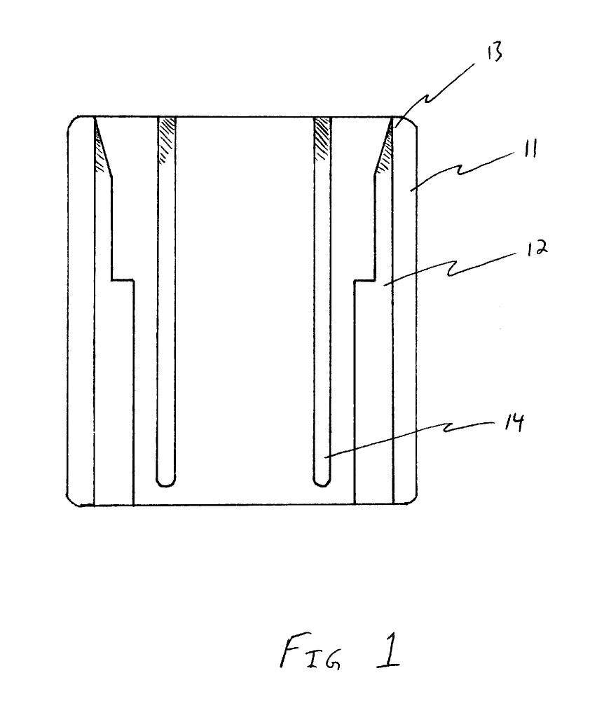

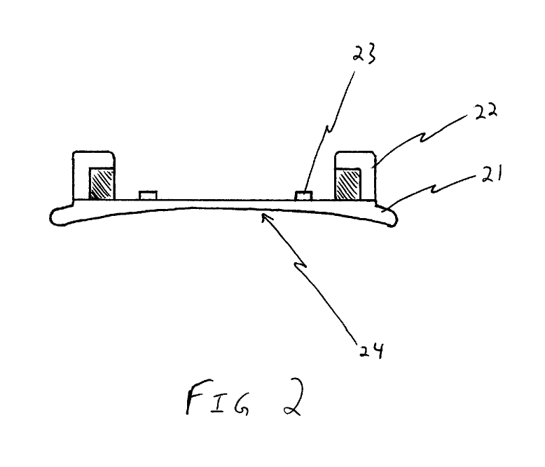

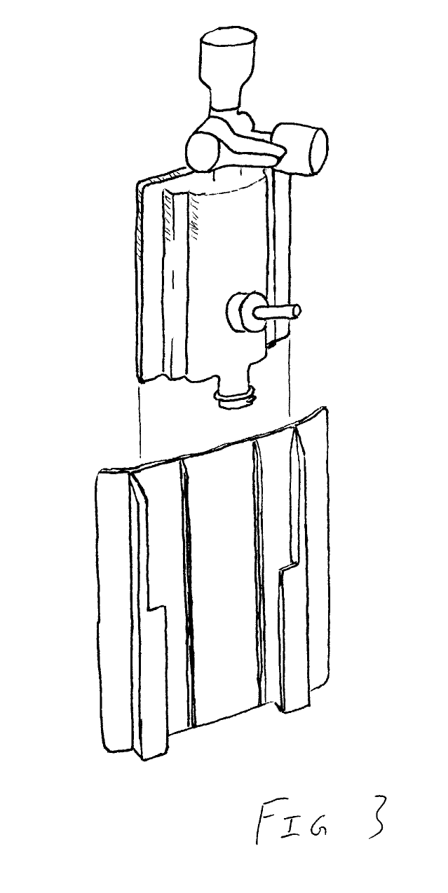

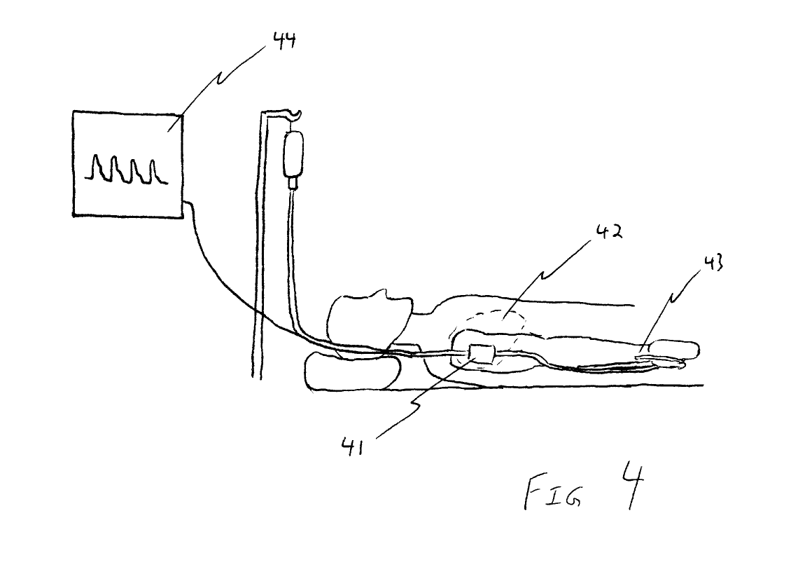

Referring to FIG. 3, the transducer slides into the holder and is held in place by the rails that were depicted in 12.

Referring to FIG. 4, the holder, 41, is applied to the desired location. In this depiction, the location is on the arm at the approximate height of the heart, 42. However, the holder can be applied to different places depending on the desired location of the holder. For illustrative purposes, the invasive catheter is depicted in 43 and the output monitor in 44.

1) A transducer holding system comprising:
(a) a base made of rigid or semi-rigid material that has the capacity to hold a transducer via sliding into place
(b) an adhesive backing to the transducer mounting device designed to be applied at the appropriate zero point for the transducer

2) A method of securing a transducer to a patient comprising of the following steps:
(a) providing a rigid or semi-rigid rectangular or square shaped base that has the capacity of holding a transducer
(b) securing the transducer in the holder via sliding in place
(c) affixing the holder to a patient, patient bed or operating table via application of adhesive backing on the transducer holder
